(19) United States
(12) Patent Application Publication
(10) Pub. No.: US 2005/0175623 A1
(43) Pub. Date: Aug. 11, 2005

(54) Saponins as anticancer agent

(76) Inventor: Zheng-Pin Wang, Ellicott, MD (US)

Correspondence Address: Zheng-Pin Wang, 3610 St. Johns Lane, Ellicott, MD, 21042, US

(21) Appl. No.: 10/775,632

(22) Filed: Feb. 10, 2004

(51) Int. Cl. C07J 17/00; A61K 31/704

(52) U.S. Cl. 424/184.1; 514/26; 536/6.1; 536/18.1

(57) ABSTRACT

The invention described herein encompasses novel discovery of anticancer agents and its compositions comprising saponins, a group of triterpenoid and steroidal saponins found in plants including Quillaja saponaria Molina (soap tree), which are used as therapeutic compounds for the treatment and prevention of cancer diseases or as a dietary supplement that offers tumor cell killing and tumor cell inhibition, and also as a anticancer potentiator with other anticancer agents.

### FIELD OF THE INVENTION

The present invention relates to discovery of novel anticancer agents and its compositions for the treatment of primary and metastatic cancers. These agents are saponins, including but not limited to, sapogenins, and its prosapogenins with one or more sugar moieties, which are found in varying levels in the bark of Quillaja saponaria Molina and other plants. The present invention claims the benefit of the Feb. 11, 2003 filing date of provisional application 60/446,281.

### THE PRIOR ART

Simply stated, saponins are molecular complexes consisting of any aglycone (sapogenin) attached to one or more sugar chains. In some cases saponins may be acylated with organic acids such as acetic, malonic, angelic and others as part of their structure (Hostettmann K. and Marston A. Saponins, Cambridge University Press, Combridge. 1995.; Rouhi A M., Chem. Eng. News 73(37):28-35, 1995.; Leung A Y., and Foster S., Encyclopedia of Common Natural Ingredients Used in Food, Drugs, and Cosmetics, 2^{nd} ed., John Wiley and Sons (Wiley-Interscience), New York (1996). These complex structures have molecular weights ranging from 600 to more than 2,000 daltons. The asymmetric distribution of their hydrophobic (aglycone) and hydrophilic (sugar) moieties confers an amphipathic character to these compounds which are largely responsible for their detergent-like properties. Saponins can be classified according to their aglycone composition as shown above: 1). Triterpene glycosides; 2). Steroid glycosides; 3). Steroid alkaloid glycosides.

The isolation of crude Quillaja saponins was reported for the first time in 1887 (Kobert, R., Arch. Exp. Pathol. Pharmakol. 23: 233-272, 1887.). Later Quillaja saponins still proved to be a complex and poorly separable mixture. Dalsgaard purified Quillaja saponins by subsequent dialysis, ion-exchane and gel filteration chromatography. He obtained a fraction, known as Quil A, which, on a weight basis, give fewer side effects and showed higher adjuvant activity. More recently, Dalsgaard et al. described the use of the bark from young tree, as opposed to that from old ones. The extract from the young trees is much less heterogeneous, and the danger of a shortage of old trees can be circumvented. Kersten et al. further fractionated Quil A by reversed-phase HPLC. 23 fractions containing saponins were isolated (Kersten, G. F. A., et al., Infection and Immunity 56: 432-438, 1988.). Kensil et al also applied RP-HPLC, the four major fractions obtained were tested for adjuvant and biological activity. Additional research was preformed on the HPLC fractions. QS-21, which is isolated from the aqueous extract of the bark by subsequent diafiltration, chromatography on silica, and preparative reversed phase chromatography (Kensil, C. R., et al., J. Immunol. 146: 431-437, 1991.; Kensil, C. R., Wu, J. Y., and Soltysik, S., In: Vaccine Design: The Subunit and Adjuvant Approach, Powell, M. F. and Newman, M. J., eds,. Plenum Press, New York. Ch. 22, 1995.). Afterward, QS-21 was seen to still consist of two components, which were separated by hydrophilic interaction liquid chromatography (Soltysik, S., Bedore, D. A., and Kensil, C. R., In: Specific Immunotherapy of Accine. Bijstijn, Ferrone, and Livingston, eds. Annals New York Acad. Sci. 690: 392-395, 1993.).

Higuchi et al. purified the components from bark by methanol extraction and column chromatography. In 1988, this group reported the first ever complete structure of a saponins from Quillaja Saponaria Molina (FIG. 1) (Higuchi, R., Tokimitsu, Y., and Komori, T., Phytochemistry 27: 1165-1168, 1988.). The Quillaja saponins are known as bidesmosides, which means that sugar moieties are attached to the aglycone at two positions. The aglycone (the genin or sapogenin) is the triterpenoid quillaic acid (3 belta, 16 alpha-dihydroxy-23-oxolean-12-en-28-oic acid, quillaja sapogenin). The sugar moieties are attached at triterpene position 3 (acetal bound), and triterpene position 28 (ester bound). Two structural features that distinguish Quillaja saponaria saponins from those of most other plant species are a fatty acid domain and a triterpene aldehyde group at position 4 (Kensil, C. R., Wu, J. Y., and Soltysik, S., In: Vaccine Design: The Subunit and Adjuvant Approach, Powell, M. F. and Newman, M. J., eds,. Plenum Press, New York. Ch. 22, 1995.). Thus far, only two complete molecular structures have been reported: QS-III (FIG. 2) from Higuchi's group and QS-21, reported by Kensil et al. (Kensil, C. R., Soltysik, S., Patel, U., and Marciani, D. J., In: Vaccines 92, Brown, F., Chanock, R. M., Ginsberg, H. S., and Lenner, R. A., eds., Cold Spring Harbor, N.Y., pp. 35-40, 1992.). However. For these structures, absolute configurations of the monosaccharides are assumed. Absolute configurations of the three chiral carbon atoms within the fatty acid moiety have not been determined. In an earlier report Higuchi and Komori use the term ‘acyl moiety’ (Higuchi, R., and Komori, T., Phytochemistry 26: 2357-2360, 1987.), while Hostettmann and Marston use the very similar term ‘acyl glycoside’ to indicate the complete 28-0 bound glycosyl moiety (Price, K. R., et al., CRC Crit. Rev. Food Sci. Nutr. 26: 27-135, 1987.).

Quillaja saponins have been discovered to have following biological activities: 1). Quillaja saponins can be lysates to human cells: Quillaja saponins in concentrations of 0.005% to 0.01% were used to permeabilize culture human intestinal epithelial cells (Jalal, F., et al., Biochem. J., 288:945, 1992.) and a 0.05% solution of saponins was used to permeabilize paraformaldehyde-saponins-fixed human fibroblasts (Hedman, K., J. Histochem. Cytochem. 28:1233, 1980.). Saponins (surface-active agents) have also been used to lyse the outer membranes of Rous sarcoma viruses, cell membranes of chicken liver and erythrocytes of human and guinea pig (Helenius, A. and von Bonsdoeff, C. H., Biochim. Biophys. Acta, 436:895, 1976.). 2). Triterpenoid group in Quillaja saponins carries the aldehyde group responsible for inducing T-cell immunity, whereas their carbohydrate moieties seem to enhance humoral immunity (perhaps by interacting with lymphocyte receptors) in a fashion similar to certain polysaccharides (Bohn J. and J. BeMiller, Carbohydrate Polymers 28:3, 1995.), and another component of quillaja saponins, the acyloyl-acyl groups, likewise appear to play a role in adjuvanticity (Kensil, C. et al., J. Immunol. 146:431, 1991.). Thus, it would be of commercial interest to develop modified Quillaja saponins which are easier to purify, potentially less toxic, chemically more stable, and with equal or better adjuvant properties than the original saponins. They have long been recognized as immune stimulators that can be used as vaccine adjuvants, (Campbell, J. B., and Peerbaye, Y. A., Res. Immunol. 143(5):526-530, 1992.), and a number of commercially available complex saponins extracts have been utilized as adjuvants (Bomford, Int. Arch. Allerg. Appl. Immun. 67:127, 1982.). 3). Applications of saponins in the anti-tumor research have been initiated in following areas: a). Saponins have been used as a potentiator of anti-tumor drugs. The results showed that the triterpene saponins jenisseensosides A, B, C, D were found to increase the accumulation and cytotoxicity of the anticancer agent cisplatin in human colon tumor cells. These compounds are glycosides of quillaic acid whose focus residue was acylated by a trans-or cis-p methoxycinnamic acid. In contrast, other saponins derivatives without this acyl moiety were not found to potentiate the accumulation and cytotoxicity of cisplatin. These results suggested the importance of the acyl moiety for activity (Gaidi G, Correia M, Chauffert B, Beltramo J L, Wagner H, Lacaille-Dubois M A. Planta Med 2002 January; 68(1):70-2). b). The effects of saponins on drug absorption has been checked through the bladder mucosa. The findings indicated that saponins were thought to be useful on intravesical chemotherapy because of increased concentration of anticancer drug (THP) in bladder tissue without that in plasma (Sasaki M, Hashimoto H, Yachiku S., Nippon Hinyokika Gakkai Zasshi 1994 September; 85(9):1353-62.). c). The triterpenoid saponins from an Australian desert tree of the Leguminosae family markedly inhibited the growth of several tumor cell lines with minimum growth inhibition in human foreskin fibroblasts, mouse fibroblasts, and immortalized breast epithelial cells at similar concentrations. The saponins induced cell cycle (G1) arrest of the human MDA-MB-453 breast cancer cell line and apoptosis of the Jurkat (T-cell leukemia) and the MDA-MB-435 breast cancer cell line. The triterpenoid saponins also partially inhibited phosphatidylinositol 3-kinase activity in Jurkat T cells in a time-dependent manner and phosphorylation in the downstream protein Akt, whereas no affect was seen on the Ras/mitogen-activated protein kinase cascade (Mujoo K, Haridas V, Hoffmann J J, Wachter G A, Hutter L K, Lu Y, Blake M E, Jayatilake G S, Bailey D, Mills G B, Gutterman J U. Cancer Res 2001 Jul. 15;61(14):5486-90.). d). Additionally, a highly potent anticancer natural saponins OSW-1 has been discovered from Omithogalum saunderside. OSW-1 has been successfully synthesized from commercially available 5-androsten-3beta-ol-17-one 79 in 10 operations with 28% overall yield. The key steps in the total synthesis included a highly regio-and stereoselective selenium dioxide-mediated allylic oxidation of 80 and a highly stereoselective 1,4-addition of alpha-alkoxy vinyl cuprates 68 to steroid 17(20)-en-16-one 12E to introduce the steroid side chain (Yu W, Jin Z. J Am Chem Soc 2002 Jun. 12;124(23):6576-83.). 4). The mechanism of chemical carcinogenesis has been explained by either a two-stage theory or a multi-stage theory which consists of initiation, promotion and progression stages (Berblum, Cancer Res., 1:807 (1941).). In these stages, the promotion stage is a long-term and reversible reaction, and the development of anti-tumor-promoters has been regarded as the most effective method for the chemoprevention of cancer. Several triterpenoid glycosides and crude drugs exhibited antitumor promoting activities on chemical carcinogenesis, and some of them strongly enhanced the inhibitory effects of other constituents. These compounds might be valuable for cancer chemoprevention by natural products (Konoshima T., In Saponins Used in Traditional and Modern Medicine Edited by Waller and yamosaki, Plenum Press, New York, 1996; P87-100.).

### SUMMARY OF THE INVENTION

The invention encompasses a discovery of novel anticancer agents, a group of plant-derived triterpenoid and steroidal saponins found in the bark of Quillaja saponaria Molina (soap tree), to a mammal in need of such therapy. In a preferred embodiment, the mammal is a human.

Saponins include but not limited to, sapogenins, and its prosapogenins with one or more sugar moieties.

Saponins, alone and in combination with other anti-cancer therapeutic agents, directly kill cancer cells through deconstruction of the cell membrane. The dosage of saponins required to induce cell membrane interruption is dependent on the type of cancer cell tested. Both cancer and normal cells, including white and red blood cells, are very sensitive to saponins at a dosage of more than 30 micrograms per ml. Cells death occur within two hours after exposure to saponins in vitro.

In present invention, saponins, alone and in combination with other anti-cancer therapeutic agents showed inhibition of cancer cell growth or proliferation through deconstruction of the cell membrane, and cell cycle arrest at G1, as well as promoting induction of specific cancer cell apoptosis. The dosage causing IC.sub.50 varies with different cancer cell lines. Jurkat T cells were highly sensitive to saponins with an IC.sub.50 of 0.48 micrograms per ml. Similarly, saponins inhibited the growth of a number of cancer cell lines with concentrations inhibiting growth by 50 percent (IC.sub.50) in the range of 0.97-15.62 micrograms per ml. The inhibitory activity of saponins, seen with cancer cells is not obvious in normal human cells such as white blood cells, hepatocytes, 3T3 cell line and fibroblasts in certain dosage ranges. Thus, the invention provides a safe dose for potent therapeutic effect without or while reducing the adverse effects on normal, healthy cells.

The differential activities of sub-fraction of quillaja saponins have been shown on cancer cell lines. In the present invention, twenty-five sub-fractions collected from Quillaja saponaria Molina (soap tree) by using preparative HPLC were tested in anticancer activity assays. QSF-I and II, including F1 to 9, didn't show obvious anticancer activity. QSF-III, including F10 to 14, possesses both of tumor cell killing and tumor cell inhibition. QSF-IV, including F 15 to 25, is more toxic and directly kills tumor cells. The anticancer activities are different between QSF-III and QSF-IV. QSF-III directly kills tumor cells when the concentration is higher, more than 60 micrograms per ml, but QSF-III inhibits cancer cell growth or proliferation when the concentration of F-III less than 50 micrograms per ml. Inhibitory activity increases with the dosage in the range of 0.2 to 30 micrograms per ml. In normal cells, QSF-III shows less toxicity than that of QSF-IV.

The tumor inhibitory effect of saponins is reversible in certain dose ranges, i.e., if the saponins are removed, tumor cells resume normal rates of growth. The cancer cells, no matter any cancer cell lines, are prominently killed by saponins when the dose is in certain ranges, for example, more than 30 micrograms per ml of culture media. In the dosage less than 15 micrograms per ml, the tumor cell killing and inhibition exhibit in dose dependence. Once saponins are removed from culture media, the new cancer cells start their proliferation and resume normal rates of growth. Tumor cells inhibited by QSF-III can mostly recovery from inhibition when the QSF-III is removed from culture medium.

The triterpen saponins can be used to enhance anticancer effects of other chemotherapeutics on tumor cells, such as colon, breast, prostate, renal, liver, pancreas and lung cancer cell lines. The triterpene saponins increase the accumulation and cytotoxicity of the anticancer agent cisplatin, 5-FU, cis-platinum, paclitaxel, mitomycin C and mizoribine in human tumor cells. Glycosides of quillaic acid act on membranes by interacting with cholesterol, plant sterols, phospholipids, and proteins. Saponins treatment is thought to break the associations between cholesterol and phospholipids, causing the formation of membrane openings resulting from small losses of cholesterol. Some of these membrane openings are transient in nature, and some are permanent after treatment with the agent Because of the transient nature of some of these membrane openings caused by saponins, saponins may be very good potentiators of anticancer chemotherapeutics.

Saponins of QSF-IV are highly toxic to mice. Ten mgs per kg of mouse body weight caused LD.sub.50 (50% lethal death). The liver toxicity reaction is major result of mouse death after injection of saponins. Three mgs per kg of body weight can be injected multiple times however, slight damage to the liver is observed under pathological microscopy. The same dosage of QSF-III (3 mg/kg body weight) doesn't show liver damage. The oral administration of saponins, both of QSF-III and IV, is not toxic to mice at any dosage used, up to 400 mg per kg of body weight. Modification of saponins preparations can developed, which reduce or eliminate the toxic effect of the fractions and components tested, and used as a pharmaceutical compositions of cancer killer potentiators and chemotherapeautics.

### BRIEF DESCRIPTION OF THE DRAWINGS

FIG. 1 demonstrates the chemical structure of QS-III isolated from Quillaja saponaria Molina by using thin-layer chromatography. The molecular formula is C₁₀₄H₁₆₈O₅₅, 8H₂O.

FIG. 2 shows the chemical structure of DS-1 isolated from Quillaja saponaria Molina by using 6% NaHCO.sub.3 and 50 percent of methanol.

FIG. 3 is chromatogram and retention times of saponins extracts of Quillaja saponaria Molina. It also shows fractions of QSF-I, QSF-II, QSF-III and QSF-IV collected from preparative high-performance liquid chromatography (HPLC). QSF-I and II include sub-fractions of F1 to F9, and QSF-III includes sub-fractions of F10 to F14, and QSF-IV integrates sub-fractions of F15 to F25.

FIGS. 4a to 4f show the activities of tumor cell killing and inhibiting at one dosage administration (7.8 micrograms per ml) of saponins in different time. 4a shows cancer cells exposed to saponins for thirty minutes at 7.8 micrograms per ml. 4b shows cancer cells (stained with Trypan Blue) killed by saponins at two-hours after exposing to saponins. 4c, d, and e show died cancer cells at 24, 48, and 72 hours after exposing to saponins, respectively. 4f shows new cancer cell proliferation 48 hours after withdrawing saponins from culture medium.

FIGS. 5a to 5d demonstrate the effects of tumor cell killing and inhibiting at 72-hours after exposing to different doses of saponins. The doses of saponins are 3.9 micrograms per ml in FIG. 5a, 1.9 micrograms per ml in FIG. 5b, 0.9 micrograms per ml in FIG. 5c and 0.4 micrograms per ml in FIG. 5d.

FIG. 6 shows cancer cell growth inhibition corresponds to the increase of saponins dosage in H-157 cancer cell culture.

FIG. 7 demonstrates activities of tumor cell killing and inhibiting by saponins with different doses and exposing time.

FIG. 8 is a flow cytometry graphics which show cancer cells exposing to saponins at 3.9 micrograms per ml for 48 hours are arrested at G1 phase of cell cycle compared to control group.

FIG. 9 demonstrates three cancer cells. Two cancer cells show green color in nucleus stained by TUNEL which represents cell apoptosis. One cancer cell shows negative staining of TUNEL and blue color in nucleus stained by DAPI for DNA. The red color represents immunocytochemical staining specific for cytokeratin 18.

FIG. 10 shows cancer cell killing and inhibiting activities in different in vitro tests using all subfractions of quillaja saponins in AsPC-1 cell line (a) and HuH-7 cell line (b). The last bar represents control test without saponins in culture medium.

FIG. 11 shows biological activities in 3T3 (a) and mouse fibroblasts (b) exposed to different subfractions of quillaja saponins in culture. The F10, F12, F13 and F14 show cancer cell inhibition in FIG. 10 and little effect on 3T3 and mouse fibroblasts. The last bar represents control test without saponins in culture medium.

### DESCRIPTION OF PREFERRED EMBODIMENTS OF THE INVENTION

Definitions

As used herein, the term “saponins” describes a class of natural products which are structurally constructed of agglycone (triterpene or steroidal) and sugars (pentose(s), hexose(s), and/or uronic acid(s)). Saponins possess anticancer activities and can be extracted from some plants, such as Quillaja Soap Trees and many Chinese Herbal Medicines.

As used herein, the term “tumor or cancer” describes a diseased state in which a carcinogenic agent or agents causes the transformation of a normal cell into an abnormal cell, the invasion of adjacent tissues by these abnormal cells, and lymphatic or blood-borne spread of malignant cells to regional lymph nodes and to distant sites, i.e., metastasis.

As used herein, the term “anticancer or anti-tumor” mean to inhibit the replication of cancer cells, to inhibit the spread of cancer, to decrease tumor size, to lessen or reduce the number of cancerous cells in vitro or in vivo.

The term “potentiator” as used herein refers to a combination which is more effective than the additive effects of any two or more single agents. A determination of a potentiation interaction between saponins, and another therapeutic agent may be based on the results obtained from the experimental tests in vitro and in vivo.

The term “membrane interruption” refers to saponins that do interfere with normal biological structures of membrane in the cells, such as tumor cells. Saponins act on membranes by interacting with cholesterol, plant sterols, phospholipids, and proteins, and causing transient holes on the membrane of cells. The cells with membrane interruption become permeated and damaged.

The term “pharmaceutical compositions” refers to an active ingredient from saponins that can be used alone or in combination with other known therapeutic agents or techniques to either improve the quality of life of the patient, or to treat cancer or solid tumors.

As used herein the term “pharmaceutically acceptable derivative” refers to any homolog, analog, or fragment corresponding to the saponins formulations as described in present invention which exhibits anti-cancer activity and is relatively non-toxic to the subject.

The term “therapeutic agent” refers to any molecule, compound or treatment that assists in the treatment of a cancer or the diseases caused thereby.

The saponins and its fragments defined herein are abbreviated as follows:

| Saponins | SP |
|---|---|
| Quillaja saponins fragment-I to II | Sub-Fractions 1 to 9 (F1 to 9) |
| Quillaja saponins fragment-III | Sub-Fractions 10 to 14 (F10-14) |
| Quillaja saponins fragment-IV | Sub-Fractions 15 to 25 (F15-25) |

### DESCRIPTION OF PREFERRED EMBODIMENTS

In accordance with the present invention, a novel anticancer agents comprises saponins, a group of plant-derived triterpenoid and steroidal saponins found in plants including in the bark of Quillaja saponaria Molina (soap tree), which are used as therapeutic compounds for the treatment of cancer or as a dietary supplement that offers tumor cell killing and tumor cell inhibition, as well as cancer prevention. In a preferred embodiment, the cancer is a human cancer.

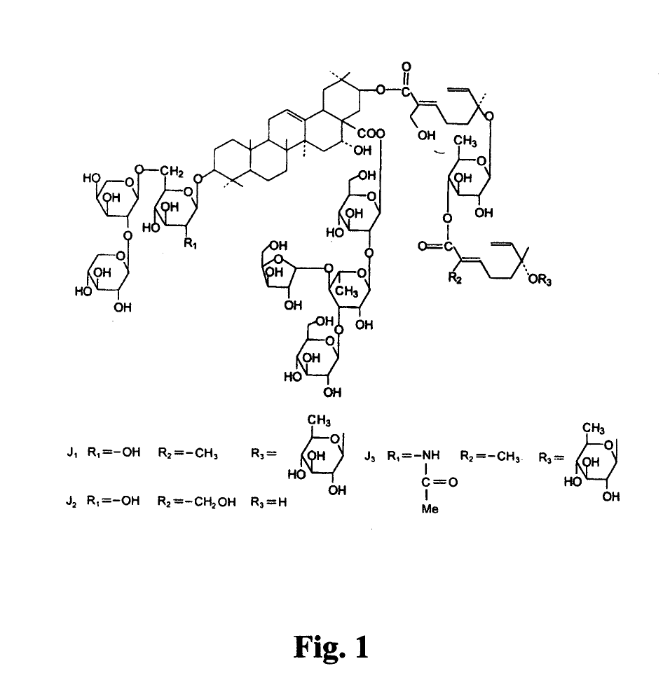

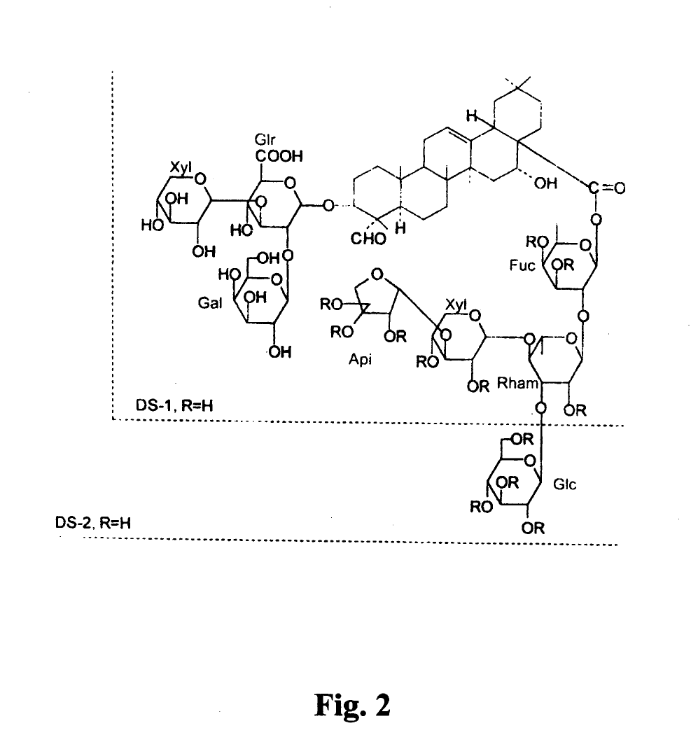

Saponins include but not limited to, sapogenins, and its prosapogenins with one or more sugar moieties. The basic chemical structures of quillaja saponins are listed in FIGS. 1 and 2 based on different extraction methods.

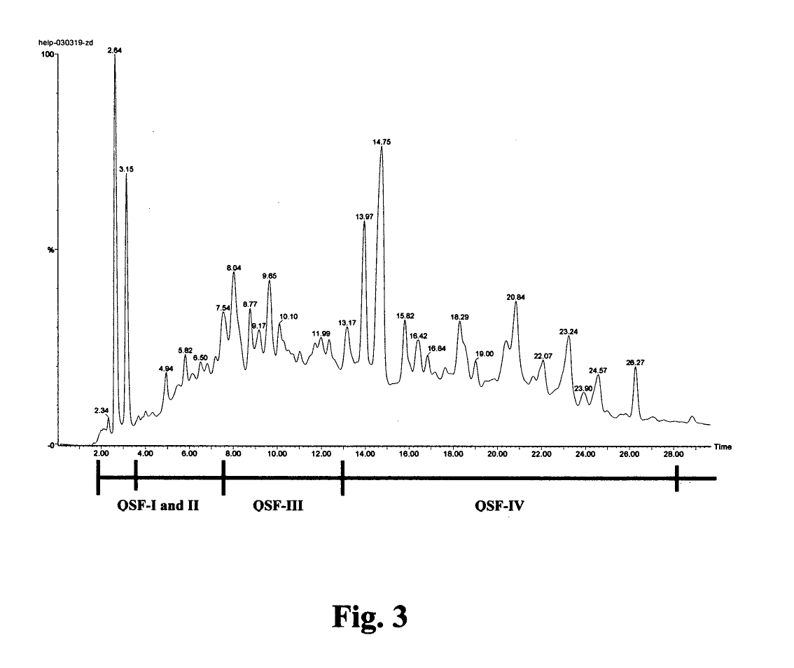

In a specific embodiment, the saponins extracted from the bark of Quillaja saponaria Molina (soap tree) comprise HPLC fractions of F1 to 25. QSF-I is collected by preparative HPLC from 2.00 min to 4.00 min, QSF-II from 4.01 min. to 7.54 min., QSF-III from 8.00 min. to 10.00 min. and QSF-IV from 12.00 min. to 27.00 min. (see FIG. 3; and Table 1).

In the anticancer activities, QSF-I and -II do not have very obviously cancer killing and inhibiting activity in vitro. QSF-III and -IV show both activities of cancer cell killing and inhibiting, QSF-III has very good dose related to cancer cell inhibiting. QSF-IV showed more tumor cell killing action that may be related to its high cytotoxicity.

In another embodiment, saponins are very toxicity to mice. Ten mgs per kg of mouse body weight cause LD.sub.50 (50% lethal death) and liver damaged by injection of saponins. Three mgs per kg of body weight can be repeatedly injected, but sleight damage of liver still excites. The same dosage of QSF-III (3 mg/kg body weight) doesn't show liver damage. The oral administration of saponins is not toxic to mice at any of dosages up to 400 mg per kg of body weight. Modification of saponins preparations can be avoided the toxicity and used as a pharmaceutical compositions of cancer killer or potentiator of chemotherapeautics.

The disclosure is based, in part, on the discovery that saponins, alone and in combination with other herbal extracts and other anti-cancer therapeutic agents, interrupt membrane proteins and its construction. The interruption of membrane proteins of cancer cells results in the tumor cell killing and tumor cell inhibiting. Saponins arrest the cell growth at G1 phase and induce apoptosis of cancer cells. The dose inducing IC.sub.50 to most cancer cell lines did lightly influence normal human hepatocytes. Thus, the invention may provide a potent therapeutic effect without or while reducing the adverse effects on normal, healthy cells.

Significantly the effect of the saponins from QSF-III and QSF-IV fractions at the dosage inducing IC.sub.50 to most cancer cell lines are reversible, i.e., if the saponins are removed, cancer cells resume normal rates of growth. Other discoveries include: (1) saponins are very stabilized material in culture media and one time administration of saponins to cancer cells in vitro plays anticancer effect for at least one hundred hours or longer. The solution of saponins stored at 4 degree centigrade for more than three months still kept anticancer effects in vitro, (2) cancer cells must be prominently killed from thirty minutes to twenty four hours after exposing to higher dosages (more than 15 micrograms per ml) of saponins, (3) cancer cells must be inhibited from growing for 24 to 48 hours before saponins-induced apoptosis occurs, and (4) when cancer cells are constantly exposed to saponins concentration of ten micrograms per ml, the cancer cells are killed by inducing apoptosis.

In accordance with the present invention, the saponins can be used alone or in combination with other known therapeutic agents or techniques to either improve the quality of life of the patient, or to treat cancer or solid tumors. The triterpen saponins can be used to enhance anticancer effects of other chemotherapeutics on tumor cells, such as colon, breast, prostate, renal, liver, pancreas and lung cancer cell lines. The triterpene saponins increase the accumulation and cytotoxicity of the anticancer agent cisplatin, 5-FU, cis-platinum, paclitaxel, mitomycin C and mizoribine in human tumor cells. Glycosides of quillaic acid act on membranes by interacting with cholesterol, plant sterols, phospholipids, and proteins. Saponins treatment is thought to break the associations between cholesterol and phospholipids, causing the formation of membrane openings resulting from small losses of cholesterol. Some of these membrane openings are transient in nature, and some are permanent after treatment with the agent. Because of the transient nature of some of these membrane openings caused by saponins, saponins may be very good potentiators of anticancer chemotherapeutics.

The following examples are illustrative of the present invention, and should not limit the scope of the invention.

Sample 1. Saponins Preparations

A. Extraction of Saponins

Two extracts of saponins from Quillaja Bark were purchased from Sigma (St. Louis, Mo.). One saponins extract (Cat. No. S 4521; Sigma) contains approximate 25% of sapogenins and another (Cat. No. S 7900; Sigma) has approximate 10% of sapogenins. Two extracts of saponins were selected through anticancer tests in vitro. It was confirmed that the anticancer effect is correspondence to the content of sapogenins in two saponins mixtures. The extract of saponins with 25% sapogenins (Cat. No. S4521; Sigma) was selected as anticancer reagent in whole anticancer tests.

B. Liquid Chromatography-Mass Spectrometry (LC-MS)

Saponins powder was dissolved in water. Ten mg of this solution was eluted on C18 (25 cm length, 4.6 mm i.d) in a linear gradient of 40% water/60% acetonitrile/0.5% formic acid to 40% water/60% acetonitrile/0.5% formic acid over 30 minutes at a 2 ml/minute flow rate. A total of four runs were made. Full liquid chromatography scanned by UV and the mass spectra of 25 major eluted peeks were recorded (see FIG. 3).

C. Preparative HPLC

Four fragments and twenty five subfractions were collected by preparative high-performance liquid chromatography (HPLC), (see Table 1).

TABLE 1
Quillaja saponins fragments (QSF) and Subfractions (F) preparative
high-performance liquid chromatography (HPLC)

| Fragments | Collection Time | Subfractions |
|---|---|---|
| QSF-I | 2.00 to 4.00 min. | F1-4 |
| QSF-II | 4.01 to 7.54 min. | F5-9 |
| QSF-III | 8.00 to 10.00 min. | F10-14 |
| QSF-IV | 12.00 to 27.00 min. | F15-25 |

Sample 2. Stanbilization Tests of Saponins' Solution

Saponins powder was dissolved in culture medium without serum and filtrated for sterilization. Solutions were stored at −20 degree centigrade and 4 degree centigrade, separately. Comparing studies for anticancer tests were conducted by using fresh preparation, one week, one month, two months and three months storage preparations at 4 degree centigrade and −20 degree centigrade.

Results:

Results didn't show any differences (P value >0.05; Student's Tests) in anticancer activity between fresh preparation and storage preparations of saponins solutions.

Sample 3: Anticancer Tests In Vitro

A. Cultivation of Cell Lines

Cells used in present invention included Hep-G2, HuH-7, HT-29, asPC-1, Renal Carcinoma Cell Line, MCF-7, TSU, DMS-53, H-157, Jurkat, Sp2/0-Ag14, 3T3, Normal Red Blood Cell (RBC), White Blood Cell (WBC), normal mouse fibroblast, and Normal human Hepatocytes from BioWhittaker (Cambrex). All cancer cell lines originated from the American Type Culture Collection (ATCC; Rockville, Md.) were seeded in the cell culture flasks with different culture media based on the instructions of ATCC based on different cell lines. Trypsinize a subconfluent monolayer culture, and collect the cells in growth medium containing serum. Centrifuge the suspension (5 min at 200 g) to pellet the cells. Resuspend the cells in growth medium and count them. Dilute the cells to 2.5-5.0×10.supper 3 cells/ml. Depending on the growth rate of the cell line and allowing 10 ml of cell suspension per microtitration plate. Transfer the cell suspension to a 96-well microplate, and, with a multichannel piptette, add 100 ul of the suspension into each well of the central 10 columns of a flat-bottomed 96-well plate (80 wells per plate), starting with column 2 and ending with column 11 and placing 0.5-1.0×10.supper.3 cells into each well. Add 100 microliters of growth medium to the eight wells in columns 1 and 12. Column 1 will be used to blank the plate reader; column 12 helps to maintain the humidity for column 11 and minimizing the “edge effect.” Put the plates in a plastic lunch box, and incubate in a humidified atmosphere at 37 degree centigrade for 1-3 d, such that the cells are in the exponential phase of growth at the time that drug is added. For nonadherent cells, prepare a suspension in fresh growth medium. Dilute the cells to 5-100×10.supper.3 cells/ml, and plate out only 100 microliters of the suspension into round-bottomed 96-well plates. Add drug immediately to these plates.

B. Cytotoxic Effect and Inhibition Assay on Tumor Cells

Generally, cells in the exponential phase of growth are exposed to saponins as an anticancer drug. The duration of exposure is usually determined as the time require for maximal damage to occur, but is also influenced by the stability of the drug. After removal of the drug, the cells are allowed to proliferate for two to three population-doubling times (PDTs) in order to distinguish between cells that remain viable and are capable of proliferation and those that remain viable but can not proliferate. The number of surviving cells is then determined indirectly by MTT dye reduction. The amount of MTT-formazan produced can be determined spectrophotometrically once the MTT-formazan has been dissolved in a suitable solvent. Incubate monolayer cultures in microtitatrion plates in a range of saponins concentrations. Remove the saponins, and feed the plates daily for two to three PDTs; then feed the plates again, and add MTT to each well. Incubate the plates in the dark for 4 h, and then remove the medium and MTT. Dissolve the water-insoluble MTT-formazan crystals in DMSO, add a buffer to adjust the final pH, and recorder the absorbance in an ELISA plate reader.

Detailed, prepare a serial twofold dilution of the saponins in growth medium to give eight concentrations. This set of concentrations should be chosen such that the highest concentration kills most of the cells and the lowest kills none of the cells. Once the toxicity of a drug is known, a smaller range of concentrations can be used. Normally, three plates are used for each drug to give triplicate determinations within one experiment. For adherent cells, remove the medium from the wells in columns 2 to 11. This can be achieved with a hypodermic needle attached to a suction line. Feed the cells in the eight wells in columns 2 and 11 with 200 microliters of fresh growth medium; these cells are the controls. Add saponins to the cells in columns 3 to 10. Only four wells are needed for each drug concentration, such that rows A-D can be used for one drug and rows E-H for a second drug. Transfer the drug solutions to 5-cm Petri dishes, and add 200 microliters to each group of four wells with a four-tip pipettor. Return the plates to the plastic box, and incubate them for a defined exposure period. For nonadherent cells, prepare the drug dilution at twice the desired final concentration, and add 100 ul to the 100 ul of cells already in the wells. At the end of the drug exposure period, remove the medium from all of the wells containing cells, and feed the cells with 100 ul of fresh medium. Centrifuge plates containing nonadherent cells (5 min at 200 g) to pellet the cells. Then remove the medium, using a fine-gauge needle to prevent disturbance of the cell pellet.

Feed the plate with 100 microliters of fresh medium at the end of the growth period, and add 50 microliters of MTT to all of the wells in columns 1 to 11. Wrap the plates in aluminum foil, and incubate them for 4 hours in a humidified atmosphere at 37 degree centigrade. Remove the medium and MTT from the wells (Centrifuge for nonadherent cells), and dissolve the remaining MTT-formazan crystals by adding 100 ul of DMSO to all of the wells in columns 1 to 11. Add glycine buffer (25 ul per well) to all of the wells containing DMSO. Record absorbance at 570 nm immediately, since the product is unstable. Use the wells in column 1, which contain medium and MTT but no cells, to blank the plate reader.

The results were also expressed as IC.sub.50 values. The median concentration of drug required to inhibit the growth of tumor cells by 50% was determined by plotting the logarithm of the drug concentration vs the growth rate (percentage of control) of the treated cells.

C. Results:

Eleven tumor cell lines and one nonadherent tumor cell lines used in present invention were highly sensitive to saponins in vitro. In series of experimental tests, saponins exhibited two very distinguished effects, tumor cell killing and tumor cell inhibition.

Tumor cell killing effect was happened within two hours after exposing cells to saponins. The tumor cell killing effect might be related to the membrane interruption of tumor cells. The dosage of saponins to directly kill cancer cells depended on the different cell lines. Jurkat, TSU and MCF-7 cell lines were the most sensitive to saponins direct killing effects, and dosage of saponins was more than 7.8 micrograms per ml. Hep G2, DMS-53, H-157, Renal Carcinoma and Myeloma cell lines were the second sensitive to the tumor cell killing effects and dosage of saponins was more than 15.6 micrograms per ml. AsPC-1, HuH-7, HT29, and WBC were directly killed when the concentration of saponins was more than 31.25 micrograms per ml which dosage was the same dosage leading hemolysis on human red blood cells.

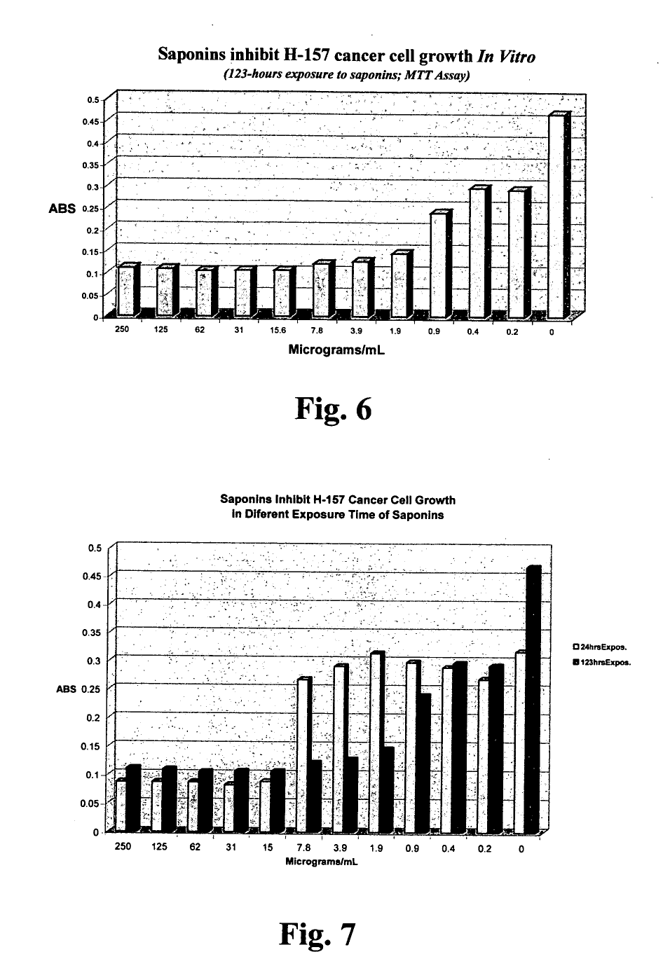

Tumor cell inhibition effect was exhibited when the cancer cell lines were exposure to saponins for 48 hours in vitro cell culture. Jurkat T cells were highly sensitive to saponins with an IC.sub.50 of 0.48 μg/ml. Similarly, saponins inhibited the growth of a number of cancer cell lines with concentrations inhibiting growth by 50 percent (IC.sub.50) in the 10 range of 0.97-15.62 μg/ml (see FIG. 6 and Table 2)

TABLE 2
Concentrations inhibiting growth by 50 percent (IC.sub.50) of Saponins

| | Saponins Dose |
|---|---|
| | (Microgram/ml) |
| Cells | in IC.sub.50 |
| Jurkat | 0.488 |
| TSU | 0.976 |
| AsPC-1 | 0.976 |
| Hep-G2 | 3.906 |
| Renal Ca. | 3.906 |
| MCF-7 | 7.812 |
| HuH-7 | 15.625 |
| DMS-53 | 15.625 |
| H-157 | 15.625 |
| HT-29 | 15.625 |
| Sp2/0-Ag14 | 15.625 |
| Human WBC | 31.250 |

The patterns for anticancer activities of cancer cell inhibition and cancer cell killing were observed through testing cancer cell growth rates after exposing saponins to cancer cells in certain time. The results showed that cancer cell killing activity could be exhibited when cancer cells were exposed to saponins within 24 hours. However, the cancer cell inhibiting activity was not obvious. Cancer cell inhibiting activity was exhibited obviously after 72-hours saponins incubation in vitro (see FIG. 7). This experiment demonstrated that the inhibition of cancer cell growth increased with the increasing of saponins dosage and exposure time in vitro.

In reversible tests, H-157 lung carcinoma cell line was used. The results showed that when cancer cells exposed to saponins with more than 31 microgrms per ml for only two hours could be killed prominently. While saponins were removed from culture medium, there was no new cancer cells proliferation any more. When cancer cells were exposed to the concentration of saponins with less than 15.6 micrograms per ml for two hours, then saponins was removed from culture medium, cancer cells started to proliferating and growing within 24 hours. Although new proliferating cancer cells could be see under microscope at the higher concentration of saponins treated cancer cells, MTT assay could not detect. The sensitivity detection of new cancer cell proliferation is much higher in microscope detection than MTT assay.

Sample 4: Abservations of Tumor Cell Killing and Inhibiting Under Microscopy.

Cancer cells, H-157 (Lung carcinoma) and RKO (Colon carcinoma) were cultured and seeded into 96-well plate for 24 hours seeding culture. Saponins were added into cell culture medium with different concentrations from 250 micrograms per ml to 0.24 microgram per ml. When cancer cells were exposed to saponins for 30 minutes, two hours, 24, 48, 96 and 123 hours, respectively. Two methods were used for detection of cancer cell membrane protein and cell death, which are immunocytochemical detection of KS1.over.4 antigen and trypan blue (0.01%, Sigma) staining. The cells were observed under microscope at each time points. One group of cancer cells was designed for reversible tests in which cancer cells were incubated in culture medium with saponins and two hours later, the saponins was aspirated and new medium without saponins was added for continuous culture.

Results:

Results from 30 minutes after exposing cancer cells to saponins showed that all cancer cells were died within 30 minutes when the concentration of saponins was more than 31 micrograms per ml. Cancer cells exposed to the dosages less than 31 micrograms per ml didn't show abnormal morphological phenotype under microscope.

Two hours later, the most of cancer cells exposing to the saponins medium with 15.6 micrograms per ml were died and while, some cells in the concentration of 7.8 micrograms per ml were died, but most cells were still survival. The cancer cells would be continuous to expose to the saponins culture medium with the dosages from 15.6 to 0.24 micrograms per ml for 24 hours. All cancer cell growth was inhibited, and inhibition grades increases with the increasing of saponins dose. The dose of 15.6 micrograms per ml caused prominent damage of all cancer cells. The observation of cancer cells in the duration from 24-hours exposure until 123-hours exposure revealed that there was a critical dose causing cancer cell prominent damage and no living cancer cell was left in whole culture wells. The 15.6 micrograms per ml is the critical dose of saponins for cancer cell killing. The dosage for IC.sub.50 of saponins is 0.98 micrograms per ml. All dosages from 15.6 to 0.24 micrograms per ml used in present invention have cancer cell inhibiting action. The saponins are very stable reagent and it was only one time administration for constantly inhibiting cancer cell growth.

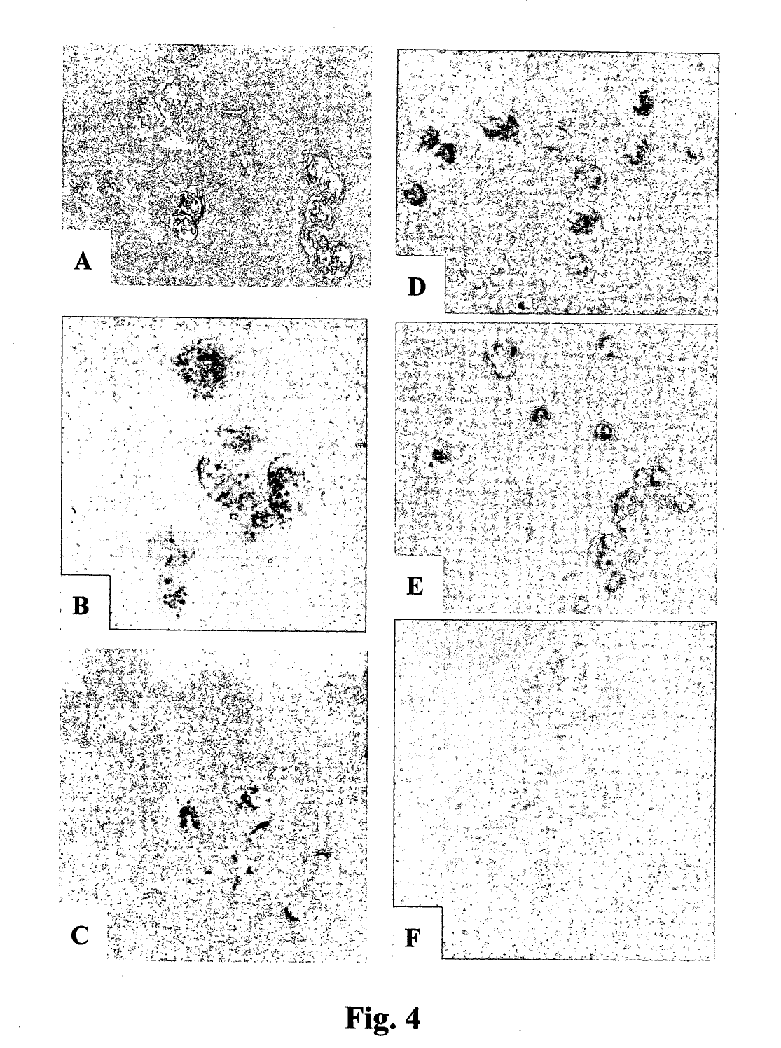

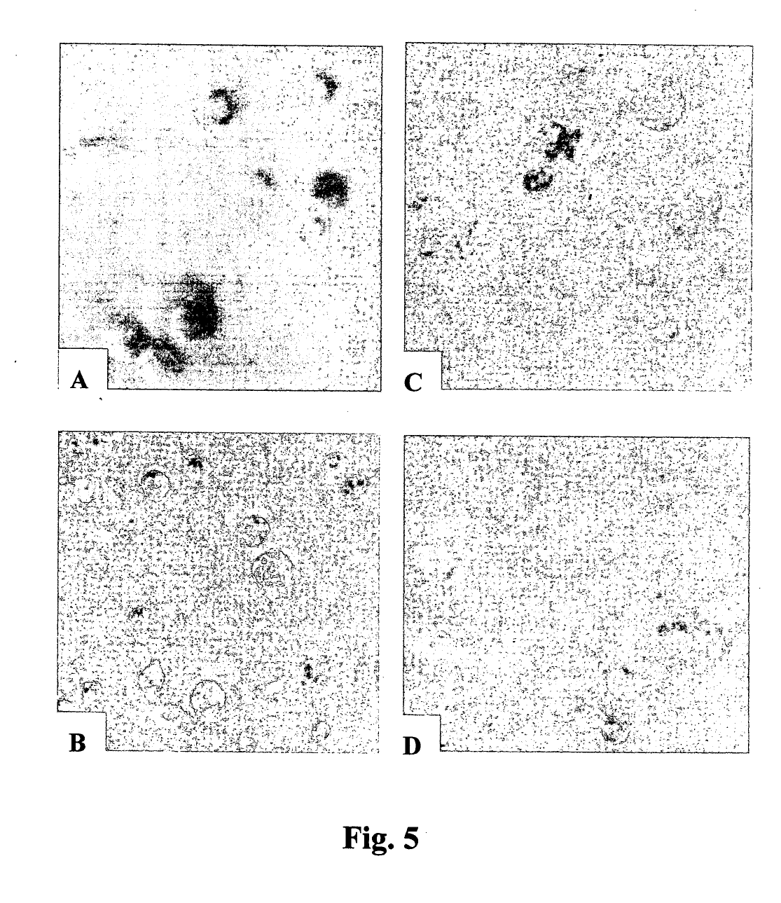

The death cancer cells in whole experiments exhibited consequences: 1). Some cancer cells died from membrane lysitic damages and signals of membrane antigen decreased or disappeared by immunocytochemical detection. These cells were detached from culture plate. and 2). Some cancer cells shriveled cell body and nucleus. The nuclei of death cancer cells were stained by Trypan blue (see FIG. 4 to 5).

Reversible tests showed when cancer cells exposed to saponins with more than 31 microgrms per ml for only two hours could be killed prominently. When saponins were removed from culture medium, there was no new cancer cells proliferation any more. When cancer cells were exposed to the concentration of saponins with less than 15.6 micrograms per ml for two hours, then saponins was removed from culture medium, cancer cells started to proliferating and growing within 24 hours. After 24 hours, the growth rate of new proliferating cancer cells recovered into normal or even over proliferation. The experiments exhibited that certain dose (more than 15 micrograms per ml) of saponins could cause cancer cell prominently damage and could not re-proliferation again.

Sample 5: Toxicity Tests in In-Vivo Experiments

A. Mice

Six-week old ICR mice of both sexes, weighing 18-22 g were used for toxicity tests. The animals were divided into four groups that are oral, muscular, peritoneal cavity administrative groups and normal control group.

B. Acute Toxicity Test-I (One Time Injection)

The groups of peritoneal, muscular and oral administration were designed into seven groups based on the dosages of saponins, respectively. The design of saponins dosages followed the four-fold dilution that was shown in table 3. In control group mice were administrated placebo medium without saponins. Each experimental and control group consisted of 6-10 mice. Saponins was dissolved in 1×PBS which was autoclaved. Injection volume was 0.5 ml for each dosage of each mouse.

The groups of oral and muscular administration of saponins had the same design as peritoneal administrative group, but only 4 mice for each group. There were ten mice injected with 0.5 ml 1×PBS in control group.

TABLE 3
Experimental design for saponins toxicity test-I

| Groups | Grp 1 | Grp 2 | Grp 3 | Grp 4 | Grp 5 | Grp 6 | Contr. |
|---|---|---|---|---|---|---|---|
| Dosages | 4,096 | 1,024 | 256 | 64 | 16 | 4 | 0 |

Grp. Group;
Dosage represents mg/kg body weight.

Results

There were the same toxicity reactions in both groups of peritoneal and muscular administration of saponins. All mice in group 1 and 2 died within 4 hrs to 6 hrs after injections of peritoneal and muscle. Before dying, mice had manifestations of nervous toxicity such as tetany and opisthotonos. Then, they were died from dyspnea and breathing stop. Animals in groups of 3, 4, and 5 died continuously within 2 to 12 hrs after injections. The manifestations after injection included hyponoia or mental retardation, and showed blue color on the skin and mucosa. The mice showed abdominal distension. Postmortem examination showed that there was some bloody leakage in peritoneal cavity and the wall of intestines occurred edema in some mice. There were no abnormal findings in the chest cavity and brain. The intestine, liver, kidney, spleen, heart, lung, and brain were collected for pathological section and examination under microscope. One mouse in group of 6 died one week later after injection and five mice were survival. The all animals in oral administrative and control groups were normally survival without any toxicity response.

C. Acute Toxicity Test-II (One Time Injection)

The groups of peritoneal administration were designed into six groups based on the dosages of saponins. The design of saponins dosages followed the two-folds dilution that was shown in table 4. In control group, mice were administrated placebo medium without saponins. Each experimental and control group consisted of five mice. Saponins was dissolved in 1×PBS which was autoclaved. Inject volume was 0.5 ml for each dosage of each mouse.

TABLE 4
Experimental design for saponins toxicity test-II

| Groups | Grp 1 | Grp 2 | Grp 3 | Grp 4 | Grp 5 | Grp 6 | Contr. |
|---|---|---|---|---|---|---|---|
| Dosages | 40 | 20 | 10 | 5 | 2.5 | 1.25 | 0 |

Grp. Group;
Dosage represents mg/kg body weight.

Results:

All animals in group 1 and 2 died within 24 hrs, and two mice in group 3 died within 24 hrs after injection of saponins. After 24 hrs, all mice in other groups were survival and no more death happened in further two weeks. LD.sub.50 (50% lethal death) dosage of saponins in mouse is 10 mg/kg body weight.

D. Acute Toxicity Test-III (Multiple Administrations of Saponins)

The groups of peritoneal administration were designed two groups which were saponins group, and control group. The dosage of saponins based on the results from experiments I and II mentioned-above was selected at 3 mg/kg body weight which is very safe dosage. In control group mice were administrated placebo medium without saponins. Each experimental and control group consisted of ten mice. Saponins was dissolved in 1×PBS which was autoclaved. Inject volume was 0.5 ml for each dosage of each mouse. The mice were kept for additional seventeen days after the last injection (see Table 5.).

TABLE 5
Experimental design for saponins toxicity test-III

| Injections | Inj. 1 | Inj. 2 | Inj. 3 | Inj. 4 | Contr. |
|---|---|---|---|---|---|
| Inj. days | Day 1 | Day 3 | Day 5 | Day 7 | Same as Saponins Grp. |

Inj. Injection;
Grp. Group;
Dosage used was 3 mg/kg body weight.

Results

The animals in both groups of saponins and control are survival very well. The growth rate in saponins group was the same as the control group in seventeen days. The blood tests were shown in Table 6.

TABLE 6
Blood examination for mice in the saponins and control groups

| Groups | Saponins group | Control Group |
|---|---|---|
| RBC | 9.03 × 10.supper.12 | 7.23 × 10.supper.12 |
| WBC | 3.90 × 10.supper.9 | 2.75 × 10.supper.9 |
| PLT | 174.00 × 10.supper.9 | 141.00 × 10.supper.9 |
| LYM | 58% | 37.9% |
| MID | 17% | 14% |
| GRA | 25% | 48.1% |

RBC: Red Blood Cell;
WBC: White Blood Cell;
PLT: Platelets;
LYM: Lymphocyte;
MID: Monocyte;
GRA: Granulocyte.

E. Toxicity Tests of Sub-Fractions of Saponins

The groups of peritoneal administration were designed three groups which were groups of QSF-III, QSF-IV and control. The dosage of sub-fractions based on the results from acute toxicity test-II mentioned-above was selected at 3 mg/kg body weight. In control group mice were administrated placebo medium without saponins. Each experimental and control group consisted of ten mice. Saponins was dissolved in 1×PBS which was autoclaved. Inject volume was 0.5 ml for each dosage of each mouse. The mice were kept for additional seventeen days after the last injection.

Results:

The animals in both groups of saponin sub-fractions and control are survival very well. The growth rate in saponins group was the same as the control group in seventeen days. In two groups, pathological indications showed that QSF-IV caused mild hepatocyte cloudy swelling under microscope, and QSF-III did not show abnormal pathological features.

F. Pathological Changes under Microscopy

The histopathological examination showed that the most prominent change was hepatocellular degenerations throughout the lobule, including cloudy swelling, hydropic degeneration or vacuolar degeneration, particularly in the animals treated by using more than 20 mgs per kg of mouse body weight. The damage was markedly decreased in 10 mgs per kg body weight group, but there was still mild cloudy swelling of hepatocytes. The batch of mice treated with 3 mgs per kg body weight and kept in survival for additional three weeks after multiple saponins administrating showed mild cloudy swelling of hepatocytes. There were no changes in liver and kidneys in the group of mice treated with 3 mgs of QSF-III per kg body weight.

Pathological changes were also found in kidney from death animals treated with high dosage of saponins (more than 20 mg per kg body weight), including glomerular congestion, cloudy swelling, granular degeneration, fibronoid degeneration and necrosis of renal tubules and hyalin pasta within tubules. The microthrombosis and hemorrhage were occasionally found in the stroma.

Sample 6: Anticancer Tests of Sub-Fractions of Quillaja Saponins

Twenty-five subfractions from F1 to F25 of quillaja saponins were prepared by preparative HPLC. Method used for anticancer tests was followed the protocol described in sample 3 and 4. Renal carcinoma cell line, HuH-7, AsPC-1, 3T3 and Mouse Fibroblasts were used in this experiment. All subfractions of saponins were used in the same dosages as the dosages used for saponins. The dosage of the highest concentration was 250 micrograms per ml and the lowest concentration was 0.24 micrograms per ml. MTT assays were purchased for detection of cancer cell killing and inhibiton.

Results:

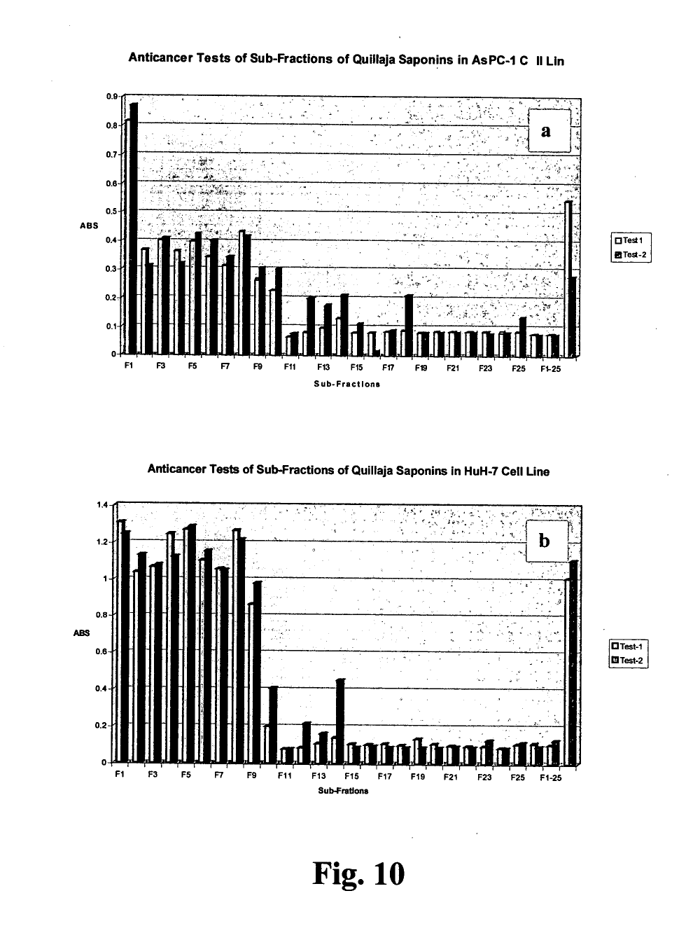

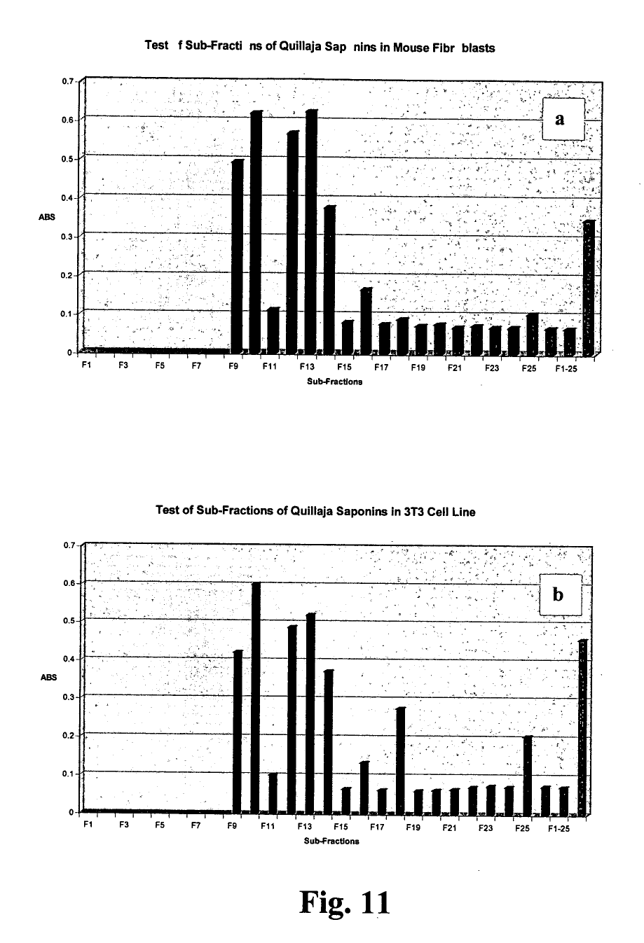

Comparing to control group, F1 to 9 didn't exhibit obvious cancer cell killing or inhibiting action. F10 to 25 showed both of killing and inhibiting actions on the cancer cells, but they had different effective actions. F 10-14 (QSF-III) could kill the cancer cells directly and very quickly when it was at higher concentration of more than 62.5 micrograms per ml. The inhibition ratio of cancer cell growth increased with the increasing of dosages from the concentration of 31.25 to 0.97 micrograms per ml. Fifty percent inhibition concentration (IC.sub.50 percent) was at seven micrograms per ml. F15-25 (QSF-IV) possessed very clear pattern of killing cancer cells (see FIG. 10). In the tests, although F10, 12, 13, and 14 showed anticancer effectiveness in FIG. 10, they didn't demonstrate growth inhibition of 3T3 and mouse fibroblast in FIG. 11.

Sample 7: Effects on the Normal Hepatocytes.

Normal hepatocytes were purchased from BioWhittaker (Cambrex). Human hepatocytes were isolated from single donors. Cells were plated on collagen-coated wells with 200,000 cells/cm.supper.2 on plates. Defined culture medium and culture conditions were followed the instruction of supplier. QSF-III being a subfraction of saponins was prepared for the tests, and doses of QSF-I were 100, 10, 5, 1, and 0.5 micrograms per ml, respectively. The cells were exposed to QSF-III for 24, 48, and 72 hours. The control cells had same culture conditions exception of QSF-III. The absorbance measurements were conducted by XTT assay.

Results:

QSF-III didn't show any statistic significance of inhibiting cell growth when the cells were exposed to above different doses and time. Only the dose of 100 micrograms per ml at 48-hour incubation group has shown inhibition activity, but no statistic significance (P value >0.05; T test) comparing with control group.

Sample 8: Anticancer Tests of Different Saponins

Two extracts of saponins from Quillaja Bark were purchased from Sigma (St. Louis, Mo.). One extract of saponins (Cat. No. S 4521; Sigma), called SP1 contains approximate 25% of sapogenins and another (Cat. No. S 7900; Sigma), called SP2 has approximate 10% of sapogenins. Two extracts of saponins were selected for the experiments. Method used for these tests was followed the methodology described in sample 3 and 4. Renal carcinoma cell line and asPC-1 were used in this experiment. Two extracts of saponins were used in the same dosages as the dosages used for saponins. The dosage of the highest concentration was 250 micrograms per ml and the lowest concentration was 0.24 micrograms per ml. MTT assays were purchased for detection of cancer cell killing and inhibition.

Result:

IC.sub.50 of SP1 was approximately 3.9 micrograms per ml in renal carcinoma cells, and 0.796 micrograms per ml in AsPC-1 cells. IC.sub.50 of SP2 was from 62.5 to 31.2 micrograms per ml in both cancer cells, respectively. The effect for killing and inhibiting cancer cells was related to the percentage of sapogenins in the extracts of Quillaja bark.

Sample 9: Cell Cycle Analysis

Renal carcinoma cells were exposed to saponins at the concentration of 3.9 micrograms per ml for 48 hours. Carefully to add 10 micro liters BrdUrd solution (1 mM BrdU in 1×DPBS) directly to each ml of tissue culture media. For this step, it is important to avoid disturbing the cells in any way that may disrupt their normal cell cycling patterns. The cell density should not exceed 2×10.supper10 cells per ml. The treated cells are then incubated for 30 minutes. The rest steps were based on the instruction of BDBrdU Flow kit. Cell cycle analysis was performed on a Becton Dickinson FACScan.

Results:

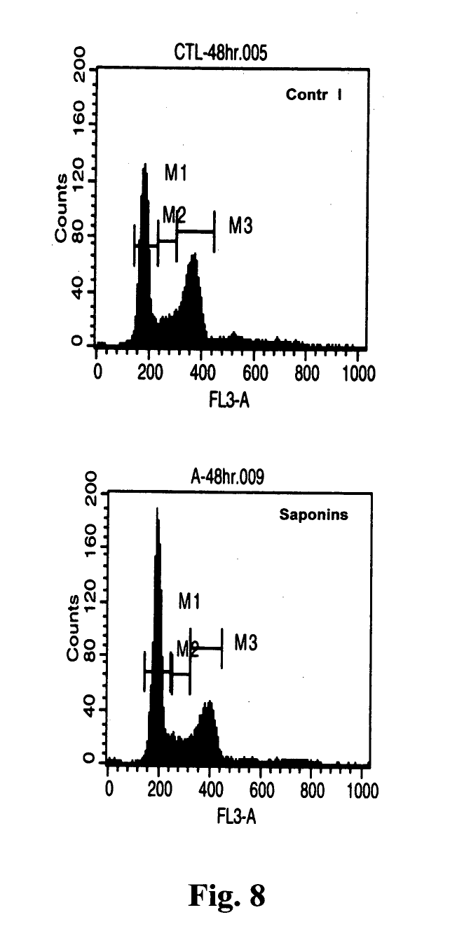

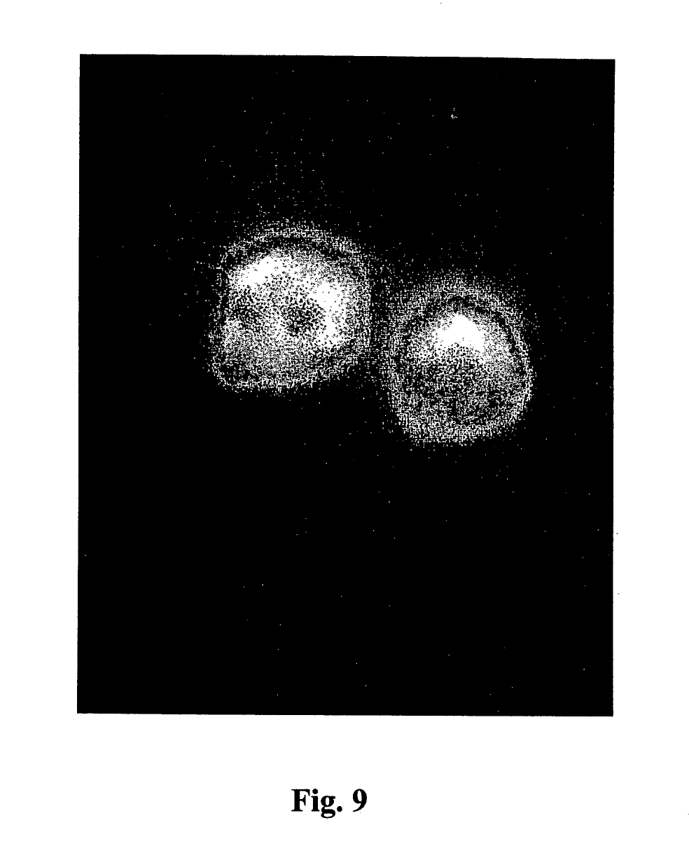

Cancer cells treated with saponins showed an increase in the population in G1 phase (56.95%) with a concomitant decrease in the percentage of cells in the S phase (9.83%) and G2 phase (28.45%), suggesting a G1 arrest. (Table 7 and FIG. 8)

TABLE 7
Cell Cycle Analysis

| Phase of | % Cells treated | % Cells |
|---|---|---|
| cell cycle | with Saponins | in Control |
| G1 | 56.95 | 42.58 |
| S | 9.83 | 10.70 |
| G2 | 28.45 | 37.60 |

Sample 10. Tunel Analysis for Detection of Cell Apoptosis

Renal carcinoma cells were exposed to saponins and QSF-III at the concentration of 3.9 micrograms per ml for 48 hours. The cancer cells were harvested from monolayer with trypsin and centrifuged down cells. Cell pellet was washed with PBS for times. Then, the cells were spun down on the slide using Cytospin and dried in air at room temperature for one hour. Cell preparations were fixed with a freshly prepared paraformadehyde solution (4% in PBS, pH 7.4) for one hour at room temperature, and then rinsed with PBS. The slides were incubated in permeabilisation solution (0.1% triton X-100 in 0.1% sodium citrate) for five minutes. The slides were rinsed tow times with PBS, and dried area around sample. Fifty microliters of TUNEL reaction mixture (Roche Molecular Biochemicals) on sample, and incubated slide in a humidified chamber for forty minutes at 37 degree centigrade. The stained slide was rinsed with PBS for tow times and dried in air. The maintaining medium with DAPI was added on the sample and sealed the sample area with cover slide. The slide was analyzed under fluorescence microscopy. Green color (FITC) was an apoptosis specific staining and blue color (DAPI) was a nucleus staining.

Results:

Fifteen to thirty percent of the cancer cells exposing to QSF-III saponins demonstrated TUNEL positive staining in their nuclei. The results suggested that cancer cell inhibiting activities of saponins be partially through inducing cell apoptosis.

### EXAMPLE 11

### Synergic Tests In Vitro

Cancer cells, H-157, were separately exposed to a combinations of saponins with concentrations of 250 to 0.2 micrograms per ml and extract of green tea with concentration of 40 micrograms per ml, or Palitaxel with concentration of 1.0×10.super.-8, or 5-FU with concentration of 1.0×10.super.-7 for 48 hours. MTT assays were purchased for detection of cancer cell inhibition.

Results:

A very significant potentiation effects were exhibited when saponins were exposed to cancer cells with other anticancer agents, crude extract of green tea, paclitaxel, and 5-FU in present invention. This potentiation effects may be related to triterpene saponins increasing the accumulation and cytotoxicity of the anticancer agents such as cisplatin, 5-FU, cis-platinum, paclitaxel, mitomycin C and mizoribine in human tumor cells. Glycosides of quillaic acid act on membranes by interacting with cholesterol, plant sterols, phospholipids, and proteins. Saponins treatment is thought to break the associations between cholesterol and phospholipids, causing the formation of membrane openings resulting from small losses of cholesterol. Some of these membrane openings are transient in nature, and some are permanent after treatment with the agent. Because of the transient nature of some of these membrane openings caused by saponins, saponins may be very good potentiators of anticancer chemotherapeutics.

1. Saponins are anticancer agents for treating and preventing mammal primary or metastatic cancer diseases.

2. The saponins of claim 1, wherein said the saponins are plant-derived saponins.

3. The saponins of claim 2, wherein said plant-derived saponins are Quillaja saponins.

4. The saponins of claim 1, wherein said the saponins include but not limited to, sapogenins, and its prosapogenins with one or more sugar moieties.

5. The Quillaja saponins of claim 3, wherein said Quillaja saponins comprise a group of saponins containing more than 25 subfractions, and being sorted into following fragments:
(a). QSF-I,
(b). QSF-II,
(c). QSF-III,
(d). QSF-IV

6. The Quillaja saponins of claim 5, wherein said QSF-I and II comprise subfractions of F1 to 9 isolated from Quillaja saponaria Molina (soap tree).

7. The Quillaja saponins of claim 5, wherein said QSF-III comprises subfractions of F10 to 14 isolated from Quillaja saponaria Molina (soap tree).

8. The Quillaja saponins of claim 5, wherein said QSF-IV comprises subfractions of F15 to 25 isolated from Quillaja saponaria Molina (soap tree).

9. The saponins of claim 1, wherein said cancer diseases are developed from:
(a). Ectodermic originated tissues
(b). Mesodermic originated tissues
(c). Endodermic originated tissues

10. The saponins of claim 1, wherein said anticancer agents are pharmaceutical compositions for treatment or prevention of cancer disease, comprising but not limited:
(a). One saponin isolated from Quillaja saponaria Molina (soap tree).
(b). Multiple saponins isolated from Quillaja saponaria Molina (soap tree).
(c). Saponin(s) in combination with other anticancer therapeutic agents.
(d). Saponin(s) in combination with biodegradable releasing chemicals.

11. The saponins of claim 1, wherein said anticancer agents are pharmaceutical formulations for treatment or prevention of cancer disease, comprising but not limited:
(a). Injection formula.
(b). Peroral formula.
(c). Spray formula.
(d). Suppository formula.
(e). Topical application or dressing formula.

12. The saponins of claim 1, wherein said anticancer agents are supplementary compositions, comprising but not limited:
(a). One saponin isolated from Quillaja saponaria Molina (soap tree).
(b). Multiple saponins isolated from Quillaja saponaria Molina (soap tree).
(c). Saponin(s) in combination with other supplementary elements

13. The saponins of claim 1, wherein said anticancer comprises but not limited:
(a). Cancer killer.
(b). Cancer inhibitor.
(c). Cancer prevention.
(d). Anticancer potentiator.

14. The anticancer of claim 13, wherein said cancer killer is that saponins can directly or indirectly kill cancer cells in vitro or in vivo.

15. The anticancer claim 13, wherein said cancer inhibitor is that saponins can directly or indirectly inhibit cancer cell growth in vitro or in vivo.

16. The anticancer of claim 13, wherein said cancer prevention is that saponins can directly or indirectly prevent cancer occurrence or reoccurrence.

17. The anticancer of claim 13, wherein said anticancer potentiator is that saponins can directly or indirectly enhance anticancer activity of other cancer therapeutic agents.

18. The closure of claim 1, wherein said mammal is human being.
